(19) United States
(12) Patent Application Publication
(10) Pub. No.: US 2026/0069458 A1
(43) Pub. Date: Mar. 12, 2026

(54) LASER BEAM APERTURE CONTROL TECHNIQUES FOR OPHTHALMIC SURGICAL EQUIPMENT

(71) Applicant: Johnson & Johnson Surgical Vision, Inc., Irvine, CA (US)

(72) Inventors: Jianming Xu, San Jose, CA (US); Frank Fellenz, Santa Clara, CA (US)

(21) Appl. No.: 18/830,466

(22) Filed: Sep. 10, 2024

(51) Int. Cl. A61F 9/008

(52) U.S. Cl. CPC A61F 9/0084; A61F 9/00823

(57) ABSTRACT

An apparatus to control laser energy for ophthalmic surgery is disclosed. The apparatus may include a laser source configured to generate a laser beam. The apparatus may also include a beam shaping apparatus having a rotatable element positioned in a path of the laser beam. The rotatable element may have a plurality of apertures and may be rotatable about an axis of rotation. Further, the apparatus may include a control unit configured to generate one or more frequency pulse trains. In addition, the apparatus may include a motor configured to rotate the rotatable element to align one of the plurality of apertures with the path of the laser beam for changing or modifying a characteristic of the laser beam. The motor may be driven based on the one or more frequency pulse trains.

### FIELD

The present disclosure generally relates to the field of ophthalmology, and in particular, to ophthalmic surgical systems and methods for achieving a desired laser pulse energy during a laser vision procedure or treatment.

### BACKGROUND

This background description is provided for the purpose of generally presenting the context of the disclosure. Unless otherwise indicated herein, material described in this section is neither expressly nor impliedly admitted to be prior art to the present disclosure or the appended claims.

Vision impairments, such as myopia (near-sightedness), hyperopia (far-sightedness) and astigmatism, can be corrected using eyeglasses or contact lenses. Alternatively, the cornea of the eye can be reshaped surgically to provide the needed optical correction. Eye surgery has become commonplace with some patients pursuing it as an elective procedure to avoid using contact lenses or glasses to correct refractive problems, and others pursuing it to correct adverse conditions such as cataracts. And, with recent developments in laser technology, laser surgery has become one of the techniques of choice for ophthalmic procedures.

Laser vision surgical systems generally employ an ultraviolet or infrared laser to remove a microscopic layer of stroml tissue from the cornea of the eye to alter the refractive characteristics of the eye. The laser removes a selected shape of the corneal tissue, often to correct refractive errors of the eye. Ultraviolet laser ablation results in photodecomposition of the corneal tissue, but generally does not cause significant thermal damage to adjacent and underlying tissues of the eye. The irradiated molecules are broken into smaller volatile fragments photochemically, directly breaking the intermolecular bonds.

Laser ablation procedures can remove the targeted stroma of the cornea to change the cornea's contour. Control over the distribution of ablation energy across the cornea may be provided by a variety of systems and methods, including the use of ablatable masks, fixed and variable apertures, attenuators, movable width-slit mechanisms, controlled scanning systems, eye movement tracking mechanisms, and the like. In some systems, the laser beam often comprises a series of discrete pulses of laser light energy, with the total shape and amount of tissue removed being determined by the shape, size, and/or location of a pattern of laser energy pulses impinging on the cornea. A variety of algorithms may be used to calculate the pattern of laser pulses used to reshape the cornea so as to correct a refractive error of the eye. The amount of pulsed laser energy at the treatment site is an important factor during a laser vision correction treatment. If the laser pulse energy does not have the desired parameters, it may be difficult to achieve the planned treatment outcome.

It would be desirable to develop improved ophthalmic surgical systems and methods to control the characteristics of laser energy for performing laser-based vision procedures and treatments.

### SUMMARY

The present disclosure is directed towards embodiments relating to systems and methods for ophthalmic laser surgery. The embodiments provide improvements for controlling characteristics of the laser energy delivered for vision correction procedures and treatments. The embodiments can be used to perform a variety of surgical procedures, such as cataract surgery, photorefractive keratectomy (PRK), phototherapeutic keratectomy (PTK), laser assisted in situ keratomileusis (LASIK), laser epithelial keratomileusis (LASEK) and the like.

The embodiments include a laser source for generating a laser beam for treating a target surface, such as a human eye. The laser beam can be directed to the target surface for forming an incision, performing tissue ablation, cauterization, and/or coagulation. A beam shaping apparatus can be used to control the characteristics of the laser beam delivered to the target surface. For example, the beam shaping apparatus may control the shape, size, diameter, profile, and/or the intensity of the laser beam. The beam shaping apparatus can include a rotatable element having a plurality of apertures of various sizes and/or shapes. The rotatable element may be rotatable or moved to align different ones of the apertures with the path of the laser beam to change or modify the characteristics of the laser beam.

The rotation of the rotatable element of the beam shaping apparatus can be controlled by a control system. The control system can include a drive mechanism, such as a stepper motor, for selectively rotating and/or moving the rotatable element to various angular orientations. By rotating the rotatable element to different positions, a selected one of the plurality of apertures can be aligned with the laser beam path for passing a portion of the laser beam through the rotatable element. The driving mechanism can be configured to quickly and accurately rotate the rotatable element to precise orientations to prevent misalignment between the selected aperture and the path of the laser beam. As a result, the driving mechanism can rapidly change the selected aperture between different laser beam pulses during a surgical procedure.

In one aspect, an apparatus for ophthalmic laser surgery is disclosed. The apparatus may include a laser source configured to generate a laser beam. The apparatus may also include a beam shaping apparatus having a rotatable element positioned in a path of the laser beam. The rotatable element may have a plurality of apertures and may be rotatable about an axis of rotation. Further, the apparatus may include a control unit configured to generate one or more frequency pulse trains. In addition, the apparatus may include a motor configured to rotate the rotatable element to align one of the plurality of apertures with the path of the laser beam for changing or modifying a characteristic of the laser beam. The motor may be driven based on the one or more frequency pulse trains.

In another aspect, a method of performing an ophthalmic surgical procedure is disclosed. The method may include rotating a rotatable element to a first orientation to align a first aperture of the rotatable element with a path of a laser beam and generating a first laser beam along the path. A portion of the first laser beam may pass through the first aperture to change a characteristic of the first laser beam pulse. The method may also include rotating the rotatable element to a second orientation to align a second aperture of the rotatable element with the path of the laser beam. The rotatable element is rotated based on one or more frequency pulse signals. Further, the method may include generating a second laser beam along the path. A portion of the second laser beam may pass through the second aperture to change a characteristic of the second laser beam. The second aperture has a different diameter or size than the first aperture.

The foregoing summary is illustrative only and is not intended to be in any way limiting. In addition to the illustrative aspects, embodiments, and features described above, further aspects, embodiments, and features will become apparent by reference to the figures and the following detailed description.

### BRIEF DESCRIPTION OF THE DRAWINGS

A more complete understanding of embodiments of the present application may be derived by referring to the detailed description and claims when considered in conjunction with the following figures, wherein like reference numbers may refer to similar elements throughout the figures. The figures are provided to facilitate understanding of the disclosure without limiting the breadth, scope, scale, or applicability of the disclosure. The drawings are not necessarily made to scale.

FIG. 1 shows a perspective view of a laser eye surgery system, in accordance with an exemplary embodiment;

FIG. 2 shows a simplified block diagram showing of configuration of a laser eye surgery system, in accordance with an exemplary embodiment;

FIG. 3 shows a simplified block diagram illustrating the configuration of a delivery assembly of a laser eye surgery system, in accordance with an exemplary embodiment;

FIG. 4 shows a beam shaping apparatus of a laser eye surgery system, in accordance with an exemplary embodiment;

FIG. 5A shows an exemplary embodiment of a rotatable element of the beam shaping apparatus of FIG. 4;

FIG. 5B shows another exemplary embodiment of a rotatable element of the beam shaping apparatus of FIG. 4;

FIG. 6 shows a control system for controlling a beam shaping apparatus of a laser eye surgery system, in accordance with an exemplary embodiment;

FIG. 7 shows a driving profile for a drive mechanism of a laser eye surgery system, in accordance with an exemplary embodiment;

FIG. 8 shows a look-up table for storing rotational data for a beam shaping apparatus, in accordance with an exemplary embodiment; and

FIG. 9 is an exemplary flow diagram illustrating a method for performing an ophthalmic surgical procedure.

### DETAILED DESCRIPTION

The figures and the following description illustrate specific exemplary embodiments. It will be appreciated that those skilled in the art will be able to devise various arrangements that, although not explicitly described or shown herein, embody the principles described herein and are included within the scope of the claims that follow this description. Furthermore, any examples described herein are intended to aid in understanding the principles of the disclosure and are to be construed as being without limitation. As a result, this disclosure is not limited to the specific embodiments or examples described below, but by the claims and their equivalents.

The present disclosure is directed towards embodiments relating to systems and methods for ophthalmic laser surgery. The embodiments provide improvements for controlling characteristics of the laser energy delivered for vision correction procedures and treatments. The embodiments can be used to perform a variety of surgical procedures, such as cataract surgery, photorefractive keratectomy (PRK), phototherapeutic keratectomy (PTK), laser assisted in situ keratomileusis (LASIK), laser epithelial keratomileusis (LASEK) and the like.

The embodiments include a laser source for generating a laser beam for treating a target surface, such as a human eye. The laser beam can be directed to the target surface for forming an incision, performing tissue ablation, cauterization, and/or coagulation. A beam shaping apparatus can be used to control the characteristics of the laser beam delivered to the target surface. For example, the beam shaping apparatus may control the shape, size, diameter, profile, and/or the intensity of the laser beam. The beam shaping apparatus can include a rotatable element having a plurality of apertures of various sizes and/or shapes. The rotatable element may be rotatable or moved to align different ones of the apertures with the path of the laser beam to change or modify the characteristics of the laser beam.

The rotation of the rotatable element of the beam shaping apparatus can be controlled by a control system. The control system can include a drive mechanism, such as a stepper motor, for selectively rotating and/or moving the rotatable element to various angular orientations. By rotating the rotatable element to different positions, a selected one of the plurality of apertures can be aligned with the laser beam path for passing a portion of the laser beam through the rotatable element. The driving mechanism can be configured to quickly and accurately rotate the rotatable element to precise orientations to prevent misalignment between the selected aperture and the path of the laser beam. As a result, the driving mechanism can rapidly change the selected aperture between different laser beam pulses during a surgical procedure.

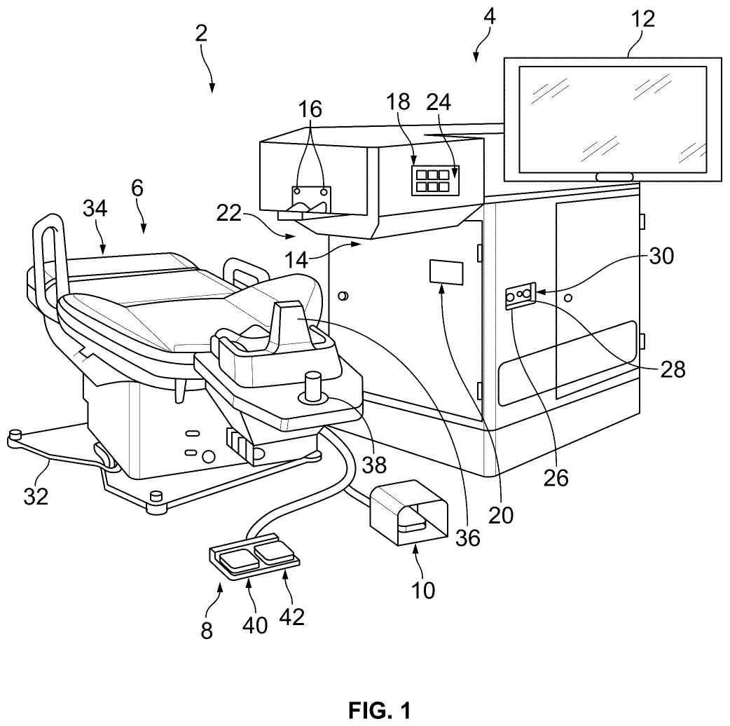

FIG. 1 shows a laser eye surgery system 2, in accordance with an exemplary embodiment, operable to form precise incisions in the cornea, in the lens capsule, and/or in the crystalline lens nucleus.

The system 2 includes a main unit 4, a patient chair 6, a dual function footswitch 8, and a laser footswitch 10.

The main unit 4 includes many primary subsystems of the system 2. For example, externally visible subsystems include a touch-screen control panel 12, a patient interface assembly 14, patient interface vacuum connections 16, a docking control keypad 18, a patient interface radio frequency identification (RFID) reader 20, external connections 22 (e.g., network, video output, footswitch, USB port, door interlock, and AC power), laser emission indicator 24, emergency laser stop button 26, key switch 28, and USB data ports 30.

The patient chair 6 includes a base 32, a patient support bed 34, a headrest 36, a positioning mechanism, and a patient chair joystick control 38 disposed on the headrest 36. The positioning control mechanism is coupled between the base 32 and the patient support bed 34 and headrest 36. The patient chair 6 is configured to be adjusted and oriented in three axes (x, y, and z) using the patient chair joystick control 38. The headrest 36 and a restrain system (not shown, e.g., a restraint strap engaging the patient's forehead) stabilize the patient's head during the procedure. The headrest 36 includes an adjustable neck support to provide patient comfort and to reduce patient head movement. The headrest 36 is configured to be vertically adjustable to enable adjustment of the patient head position to provide patient comfort and to accommodate variation in patient head size.

The patient chair 6 allows for tilt articulation of the patient's legs, torso, and head using manual adjustments. The patient chair 6 accommodates a patient load position, a suction ring capture position, and a patient treat position. In the patient load position, the chair 6 is rotated out from under the main unit 4 with the patient chair back in an upright position and patient footrest in a lowered position. In the suction ring capture position, the chair is rotated out from under the main unit 4 with the patient chair back in reclined position and patient footrest in raised position. In the patient treat position, the chair is rotated under the main unit 4 with the patient chair back in reclined position and patient footrest in raised position.

The patient chair 6 is equipped with a “chair enable” feature to protect against unintended chair motion. The patient chair joystick control 38 can be enabled in either of two ways. First, the patient chair joystick control 38 incorporates a “chair enable” button located on the top of the joystick. Control of the position of the patient chair 6 via the joystick 38 can be enabled by continuously pressing the “chair enable” button. Alternately, the left foot switch 40 of the dual function footswitch 8 can be continuously depressed to enable positional control of the patient chair 6 via the joystick 38.

In many embodiments, the patient chair joystick control 38 is a proportional controller. For example, moving the joystick a small amount can be used to cause the chair to move slowly. Moving the joystick a large amount can be used to cause the chair to move faster. Holding the joystick at its maximum travel limit can be used to cause the chair to move at the maximum chair speed. The available chair speed can be reduced as the patient approaches the patient interface assembly 14.

The emergency stop button 26 can be pushed to stop emission of all laser output, release vacuum that couples the patient to the system 2, and disable the patient chair 6. The stop button 26 is located on the system front panel, next to the key switch 28.

The key switch 28 can be used to enable the system 2. When in a standby position, the key can be removed and the system is disabled. When in a ready position, the key enables power to the system 2.

The dual function footswitch 8 is a dual footswitch assembly that includes the left foot switch 40 and a right foot switch 42. The left foot switch 40 is the “chair enable” footswitch. The right foot switch 42 is a “vacuum ON” footswitch that enables vacuum to secure a liquid optics interface suction ring to the patient's eye. The laser footswitch 10 is a shrouded footswitch that activates the treatment laser when depressed while the system is enabled.

In many embodiments, the system 2 includes external communication connections. For example, the system 2 can include a network connection (e.g., an RJ45 network connection) for connecting the system 2 to a network. The network connection can be used to enable network printing of treatment reports, remote access to view system performance logs, and remote access to perform system diagnostics.

The system 2 can include a video output port (e.g., HDMI) that can be used to output video of treatments performed by the system 2. The output video can be displayed on an external monitor for, for example, viewing by family members and/or training. The output video can also be recorded for, for example, archival purposes. The system 2 can include one or more data output ports (e.g., USB) to, for example, enable export of treatment reports to a data storage device. The treatments reports stored on the data storage device can then be accessed at a later time for any suitable purpose such as, for example, printing from an external computer in the case where the user without access to network based printing.

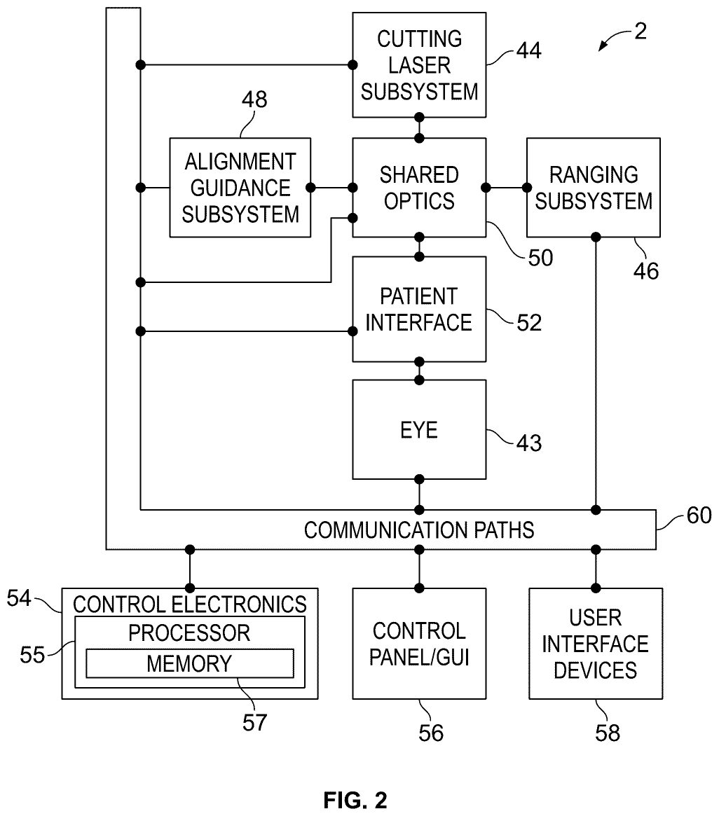

FIG. 2 shows a simplified block diagram of the system 2 coupled with a patient's eye 43. The patient's eye 43 comprises a cornea, a lens and an iris. The iris defines a pupil of the eye 43 that may be used for alignment of eye 43 with system 2. The system 2 includes a cutting laser subsystem 44, a ranging subsystem 46, an alignment guidance subsystem 48, shared optics 50, a patient interface 52, control electronics 54, a control panel/GUI 56, user interface devices 58, and communication paths 60. The control electronics 54 is operatively coupled via the communication paths 60 with the cutting laser subsystem 44, the ranging subsystem 46, the alignment guidance subsystem 48, the shared optics 50, the patient interface 52, the control panel/GUI 56, and the user interface devices 58.

In many embodiments, the cutting laser subsystem 44 incorporates femtosecond (FS) laser technology. By using femtosecond laser technology, a short duration (e.g., approximately 10⁻¹³ seconds in duration) laser pulse (with energy level in the micro joule range) can be delivered to a tightly focused point to disrupt tissue, thereby substantially lowering the energy level required as compared to the level required for ultrasound fragmentation of the lens nucleus and as compared to laser pulses having longer durations.

The cutting laser subsystem 44 can produce laser pulses having a wavelength suitable to the configuration of the system 2. As a non-limiting example, the system 2 can be configured to use a cutting laser subsystem 44 that produces laser pulses having a wavelength from 1020 nm to 1050 nm. For example, the cutting laser subsystem 44 can have a diode-pumped solid-state configuration with a 1030 (+/−5) nm center wavelength.

The cutting laser subsystem 44 can include control and conditioning components. For example, such control components can include components such as a beam attenuator to control the energy of the laser pulse and the average power of the pulse train, a fixed aperture to control the cross-sectional spatial extent of the beam containing the laser pulses, one or more power monitors to monitor the flux and repetition rate of the beam train and therefore the energy of the laser pulses, and a shutter to allow/block transmission of the laser pulses. Such conditioning components can include an adjustable zoom assembly to adapt the beam containing the laser pulses to the characteristics of the system 2 and a fixed optical relay to transfer the laser pulses over a distance while accommodating laser pulse beam positional and/or directional variability, thereby providing increased tolerance for component variation.

The ranging subsystem 46 is configured to measure the spatial disposition of eye structures in three dimensions. The measured eye structures can include the anterior and posterior surfaces of the cornea, the anterior and posterior portions of the lens capsule, the iris, and the limbus. In many embodiments, the ranging subsystem 46 utilizes optical coherence tomography (OCT) imaging. As a non-limiting example, the system 2 can be configured to use an OCT imaging system employing wavelengths from 780 nm to 970 nm. For example, the ranging subsystem 46 can include an OCT imaging system that employs a broad spectrum of wavelengths from 810 nm to 850 nm. Such an OCT imaging system can employ a reference path length that is adjustable to adjust the effective depth in the eye of the OCT measurement, thereby allowing the measurement of system components including features of the patient interface that lie anterior to the cornea of the eye and structures of the eye that range in depth from the anterior surface of the cornea to the posterior portion of the lens capsule and beyond.

The alignment guidance subsystem 48 can include a laser diode or gas laser that produces a laser beam used to align optical components of the system 2. The alignment guidance subsystem 48 can include LEDs or lasers that produce a fixation light to assist in aligning and stabilizing the patient's eye during docking and treatment. The alignment guidance subsystem 48 can include a laser or LED light source and a detector to monitor the alignment and stability of the actuators used to position the beam in X, Y, and Z. The alignment guidance subsystem 48 can include a video system that can be used to provide imaging of the patient's eye to facilitate docking of the patient's eye 43 to the patient interface 52. The imaging system provided by the video system can also be used to direct via the GUI the location of cuts. The imaging provided by the video system can additionally be used during the laser eye surgery procedure to monitor the progress of the procedure, to track movements of the patient's eye 43 during the procedure, and to measure the location and size of structures of the eye such as the pupil and/or limbus.

The shared optics 50 provides a common propagation path that is disposed between the patient interface 52 and each of the cutting laser subsystem 44, the ranging subsystem 46, and the alignment guidance subsystem 48. In many embodiments, the shared optics 50 includes beam combiners to receive the emission from the respective subsystem (e.g., the cutting laser subsystem 44, and the alignment guidance subsystem 48) and redirect the emission along the common propagation path to the patient interface. In many embodiments, the shared optics 50 includes an objective lens assembly that focuses each laser pulse into a focal point. In many embodiments, the shared optics 50 includes scanning mechanisms operable to scan the respective emission in three dimensions. For example, the shared optics can include an XY-scan mechanism(s) and a Z-scan mechanism. The XY-scan mechanism(s) can be used to scan the respective emission in two dimensions transverse to the propagation direction of the respective emission. The Z-scan mechanism can be used to vary the depth of the focal point within the eye 43. In many embodiments, the scanning mechanisms are disposed between the laser diode and the objective lens such that the scanning mechanisms are used to scan the alignment laser beam produced by the laser diode. In contrast, in many embodiments, the video system is disposed between the scanning mechanisms and the objective lens such that the scanning mechanisms do not affect the image obtained by the video system.

The patient interface 52 is used to restrain the position of the patient's eye 43 relative to the system 2. In many embodiments, the patient interface 52 employs a suction ring that is vacuum attached to the patient's eye 43. The suction ring is then coupled with the patient interface 52, for example, using vacuum to secure the suction ring to the patient interface 52. In many embodiments, the patient interface 52 includes an optically transmissive structure having a posterior surface that is displaced vertically from the anterior surface of the patient's cornea and a region of a suitable liquid (e.g., a sterile buffered saline solution (BSS) such as Alcon BSS (Alcon Part Number 351-55005-1) or equivalent) is disposed between and in contact with the patient interface lens posterior surface and the patient's cornea and forms part of a transmission path between the shared optics 50 and the patient's eye 43. The optically transmissive structure may comprise a lens 96 having one or more curved surfaces. Alternatively, the patient interface 52 may comprise an optically transmissive structure having one or more substantially flat surfaces such as a parallel plate or wedge. In many embodiments, the patient interface lens is disposable and can be replaced at any suitable interval, such as before each eye treatment.

The control electronics 54 controls the operation of and can receive input from the cutting laser subsystem 44, the ranging subsystem 46, the alignment guidance subsystem 48, the patient interface 52, the control panel/GUI 56, and the user interface devices 58 via the communication paths 60. The communication paths 60 can be implemented in any suitable configuration, including any suitable shared or dedicated communication paths between the control electronics 54 and the respective system components. The control electronics 54 can include any suitable components, such as one or more processor, one or more field-programmable gate array (FPGA), and one or more memory storage devices. In many embodiments, the control electronics 54 controls the control panel/GUI 56 to provide for pre-procedure planning according to user specified treatment parameters as well as to provide user control over the laser eye surgery procedure.

The user interface devices 58 can include any suitable user input device suitable to provide user input to the control electronics 54. For example, the user interface devices 58 can include devices such as, for example, the dual function footswitch 8, the laser footswitch 10, the docking control keypad 18, the patient interface radio frequency identification (RFID) reader 20, the emergency laser stop button 26, the key switch 28, and the patient chair joystick control 38.

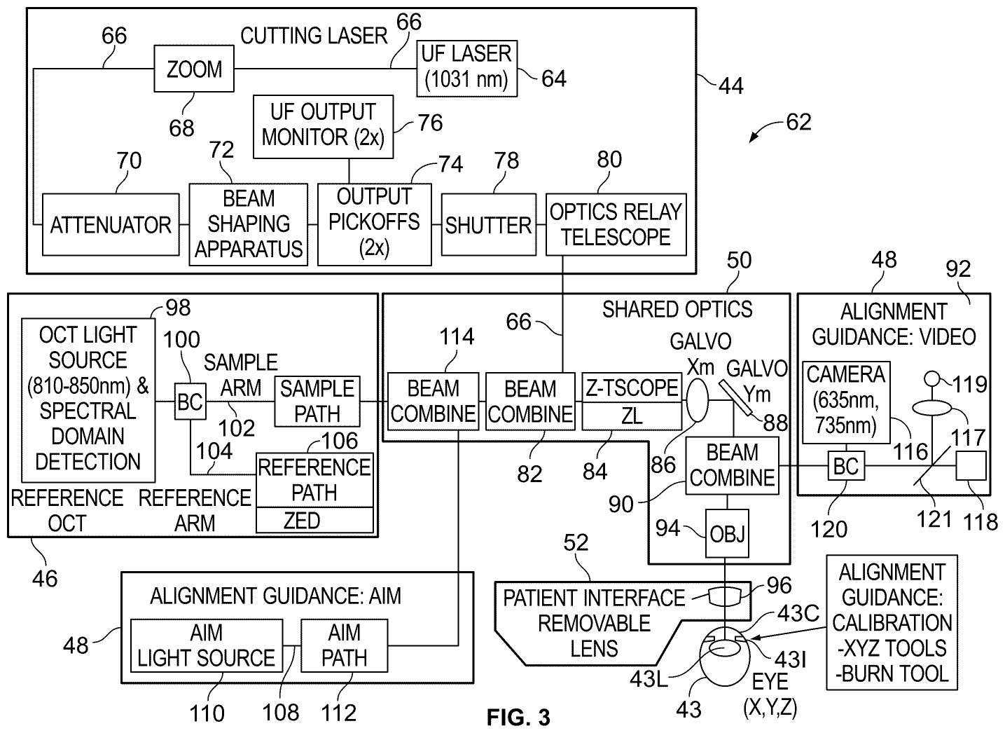

FIG. 3 is a simplified block diagram illustrating an assembly 62, in accordance with an exemplary embodiment, that can be included in the system 2. The assembly 62 is a non-limiting example of suitable configurations and integration of the cutting laser subsystem 44, the ranging subsystem 46, the alignment guidance subsystem 48, the shared optics 50, and the patient interface 52. Other configurations and integration of the cutting laser subsystem 44, the ranging subsystem 46, the alignment guidance subsystem 48, the shared optics 50, and the patient interface 52 may be possible and may be apparent to a person of skill in the art.

The assembly 62 is operable to project and scan optical beams into the patient's eye 43. The cutting laser subsystem 44 includes an ultrafast (UF) laser 64 (e.g., a femtosecond laser). Using the assembly 62, optical beams can be scanned in the patient's eye 43 in three dimensions: X, Y, Z. For example, short-pulsed laser light generated by the UF laser 64 can be focused into eye tissue to produce dielectric breakdown to cause photodisruption around the focal point (the focal zone), thereby rupturing the tissue in the vicinity of the photo-induced plasma. In the assembly 62, the wavelength of the laser light can vary between 800 nm to 1200 nm and the pulse width of the laser light can vary from 10 fs to 10000 fs. The pulse repetition frequency can also vary from 10 kHz to 500 kHz. Safety limits with regard to unintended damage to non-targeted tissue bound the upper limit with regard to repetition rate and pulse energy. Threshold energy, time to complete the procedure, and stability can bound the lower limit for pulse energy and repetition rate. The peak power of the focused spot in the eye 43 and specifically within the crystalline lens and the lens capsule of the eye is sufficient to produce optical breakdown and initiate a plasma-mediated ablation process. Near-infrared wavelengths for the laser light are preferred because linear optical absorption and scattering in biological tissue is reduced for near-infrared wavelengths. As an example, the UF laser 64 can be a repetitively pulsed 1031 nm device that produces pulses with less than 600 fs duration at a repetition rate of 120 kHz (+/−5%) and individual pulse energy in the 1 to 20 micro joule range.

The cutting laser subsystem 44 is controlled by the control electronics 54 and the user, via the control panel/GUI 56 and the user interface devices 58, to create a laser pulse beam 66. The control panel/GUI 56 is used to set system operating parameters, process user input, display gathered information such as images of ocular structures, and display representations of incisions to be formed in the patient's eye 43.

The generated laser pulse beam 66 proceeds through a zoom assembly 68. The laser pulse beam 66 may vary from unit to unit, particularly when the UF laser 64 may be obtained from different laser manufacturers. For example, the beam diameter of the laser pulse beam 66 may vary from unit to unit (e.g., by +/−20%). The beam may also vary with regard to beam quality, beam divergence, beam spatial circularity, and astigmatism. In many embodiments, the zoom assembly 68 is adjustable such that the laser pulse beam 66 exiting the zoom assembly 68 has consistent beam diameter and divergence unit to unit.

After exiting the zoom assembly 68, the laser pulse beam 66 proceeds through an attenuator 70. The attenuator 70 is used to adjust the transmission of the laser beam and thereby the energy level of the laser pulses in the laser pulse beam 66. The attenuator 70 is controlled via the control electronics 54.

After exiting the attenuator 70, the laser pulse beam 66 proceeds through beam shaping or spot control apparatus 72. The beam shaping apparatus includes one or more apertures. The apertures set the outer useful diameter of the laser pulse beam 66. In turn the zoom determines the size of the beam at the selected aperture location and therefore the amount of light that is transmitted. The amount of transmitted light is bounded both high and low. The upper is bounded by the requirement to achieve the highest numerical aperture achievable in the eye. High NA promotes low threshold energies and greater safety margin for untargeted tissue. The lower is bound by the requirement for high optical throughput. Too much transmission loss in the system shortens the lifetime of the system as the laser output and system degrades over time. Additionally, consistency in the transmission through the apertures promotes stability in determining optimum settings (and sharing of) for each procedure. Typically to achieve optimal performance the transmission through one of the apertures is set to be between 88% to 92%.

After exiting the beam shaping apparatus 72, the laser pulse beam 66 proceeds through two output pickoffs 74. Each output pickoff 74 can include a partially reflecting mirror to divert a portion of each laser pulse to a respective output monitor 76. Two output pickoffs 74 (e.g., a primary and a secondary) and respective primary and secondary output monitors 76 are used to provide redundancy in case of malfunction of the primary output monitor 76.

After exiting the output pickoffs 74, the laser pulse beam 66 proceeds through a system-controlled shutter 78. The system-controlled shutter 78 ensures on/off control of the laser pulse beam 66 for procedural and safety reasons. The two output pickoffs precede the shutter allowing for monitoring of the beam power, energy, and repetition rate as a pre-requisite for opening the shutter.

After exiting the system-controlled shutter 78, the optical beam proceeds through an optics relay telescope 80. The optics relay telescope 80 propagates the laser pulse beam 66 over a distance while accommodating positional and/or directional variability of the laser pulse beam 66, thereby providing increased tolerance for component variation. As an example, the optical relay can be a keplerian afocal telescope that relays an image of the aperture position to a conjugate position near to the XY galvo mirror positions. In this way, the position of the beam at the XY galvo location is invariant to changes in the beams angle at the aperture position. Similarly the shutter does not have to precede the relay and may follow after or be included within the relay.

After exiting the optics relay telescope 80, the laser pulse beam 66 is transmitted to the shared optics 50, which propagates the laser pulse beam 66 to the patient interface 52. The laser pulse beam 66 is incident upon a beam combiner 82, which reflects the laser pulse beam 66 while transmitting optical beams from the ranging subsystem 46 and the alignment guidance subsystem 48: AIM.

Following the beam combiner 82, the laser pulse beam 66 continues through a Z-telescope 84, which is operable to scan focus position of the laser pulse beam 66 in the patient's eye 43 along the Z axis. For example, the Z-telescope 84 can include a Galilean telescope with two lens groups (each lens group includes one or more lenses). One of the lens groups moves along the Z axis about the collimation position of the Z-telescope 84. In this way, the focus position of the spot in the patient's eye 43 moves along the Z axis. In general, there is a relationship between the motion of lens group and the motion of the focus point. For example, the Z-telescope can have an approximate 2× beam expansion ratio and close to a 1:1 relationship of the movement of the lens group to the movement of the focus point. The exact relationship between the motion of the lens and the motion of the focus in the z axis of the eye coordinate system does not have to be a fixed linear relationship. The motion can be nonlinear and directed via a model or a calibration from measurement or a combination of both. Alternatively, the other lens group can be moved along the Z axis to adjust the position of the focus point along the Z axis. The Z-telescope 84 functions as z-scan device for scanning the focus point of the laser pulse beam 66 in the patient's eye 43. The Z-telescope 84 can be controlled automatically and dynamically by the control electronics 54 and selected to be independent or to interplay with the X and Y scan devices described next.

After passing through the Z-telescope 84, the laser pulse beam 66 is incident upon an X-scan device 86, which is operable to scan the laser pulse beam 66 in the X direction, which is dominantly transverse to the Z axis and transverse to the direction of propagation of the laser pulse beam 66. The X-scan device 86 is controlled by the control electronics 54, and can include suitable components, such as a motor, galvanometer, or any other well-known optic moving device. The relationship of the motion of the beam as a function of the motion of the X actuator does not have to be fixed or linear. Modeling or calibrated measurement of the relationship or a combination of both can be determined and used to direct the location of the beam.

After being directed by the X-scan device 86, the laser pulse beam 66 is incident upon a Y-scan device 88, which is operable to scan the laser pulse beam 66 in the Y direction, which is dominantly transverse to the X and Z axes. The Y-scan device 88 is controlled by the control electronics 54, and can include suitable components, such as a motor, galvanometer, or any other well-known optic moving device. The relationship of the motion of the beam as a function of the motion of the Y actuator does not have to be fixed or linear. Modeling or calibrated measurement of the relationship or a combination of both can be determined and used to direct the location of the beam. Alternatively, the functionality of the X-scan device 86 and the Y-scan device 88 can be provided by an XY-scan device configured to scan the laser pulse beam 66 in two dimensions transverse to the Z axis and the propagation direction of the laser pulse beam 66. The X-scan and Y-scan devices 86, 88 change the resulting direction of the laser pulse beam 66, causing lateral displacements of UF focus point located in the patient's eye 43.

After being directed by the Y-scan device 88, the laser pulse beam 66 passes through a beam combiner 90. The beam combiner 90 is configured to transmit the laser pulse beam 66 while reflecting optical beams to and from a video subsystem 92 of the alignment guidance subsystem 48.

After passing through the beam combiner 90, the laser pulse beam 66 passes through an objective lens assembly 94. The objective lens assembly 94 can include one or more lenses. In many embodiments, the objective lens assembly 94 includes multiple lenses. The complexity of the objective lens assembly 94 may be driven by the scan field size, the focused spot size, the degree of telecentricity, the available working distance on both the proximal and distal sides of objective lens assembly 94, as well as the amount of aberration control.

After passing through the objective lens assembly 94, the laser pulse beam 66 passes through the patient interface 52. As described above, in many embodiments, the patient interface 52 includes a patient interface lens 96 having a posterior surface that is displaced vertically from the anterior surface of the patient's cornea and a region of a suitable liquid (e.g., a sterile buffered saline solution (BSS) such as Alcon BSS (Alcon Part Number 351-55005-1) or equivalent) is disposed between and in contact with the posterior surface of the patient interface lens 96 and the patient's cornea and forms part of an optical transmission path between the shared optics 50 and the patient's eye 43.

The shared optics 50 under the control of the control electronics 54 can automatically generate aiming, ranging, and treatment scan patterns. Such patterns can be comprised of a single spot of light, multiple spots of light, a continuous pattern of light, multiple continuous patterns of light, and/or any combination of these. In addition, the aiming pattern (using the aim beam 108 described below) need not be identical to the treatment pattern (using the laser pulse beam 66), but can optionally be used to designate the boundaries of the treatment pattern to provide verification that the laser pulse beam 66 will be delivered only within the desired target area for patient safety. This can be done, for example, by having the aiming pattern provide an outline of the intended treatment pattern. This way the spatial extent of the treatment pattern can be made known to the user, if not the exact locations of the individual spots themselves, and the scanning thus optimized for speed, efficiency, and/or accuracy. The aiming pattern can also be made to be perceived as blinking in order to further enhance its visibility to the user. Likewise, the ranging beam need not be identical to the treatment beam or pattern. The ranging beam needs only to be sufficient enough to identify targeted surfaces. These surfaces can include the cornea and the anterior and posterior surfaces of the lens and may be considered spheres with a single radius of curvature. Also the optics shared by the alignment guidance: video subsystem does not have to be identical to those shared by the treatment beam. The positioning and character of the laser pulse beam 66 and/or the scan pattern the laser pulse beam 66 forms on the eye 43 may be further controlled by use of an input device such as a joystick, or any other appropriate user input device (e.g., control panel/GUI 56) to position the patient and/or the optical system.

The control electronics 54 can be configured to target the targeted structures in the eye 43 and ensure that the laser pulse beam 66 will be focused where appropriate and not unintentionally damage non-targeted tissue. Imaging modalities and techniques described herein, such as those mentioned above, or ultrasound may be used to determine the location and measure the thickness of the lens and lens capsule to provide greater precision to the laser focusing methods, including 2D and 3D patterning. Laser focusing may also be accomplished by using one or more methods including direct observation of an aiming beam, or other known ophthalmic or medical imaging modalities, such as those mentioned above, and/or combinations thereof. Additionally the ranging subsystem such as an OCT can be used to detect features or aspects involved with the patient interface. Features can include fiducials places on the docking structures and optical structures of the disposable lens such as the location of the anterior and posterior surfaces.

In the embodiment of FIG. 3, the ranging subsystem 46 includes an OCT imaging device. Additionally or alternatively, imaging modalities other than OCT imaging can be used. An OCT scan of the eye can be used to measure the spatial disposition (e.g., three dimensional coordinates such as X, Y, and Z of points on boundaries) of structures of interest in the patient's eye 43. Such structure of interest can include, for example, the anterior surface of the cornea, the posterior surface of the cornea, the anterior portion of the lens capsule, the posterior portion of the lens capsule, the anterior surface of the crystalline lens, the posterior surface of the crystalline lens, the iris, the pupil, and/or the limbus. The spatial disposition of the structures of interest and/or of suitable matching geometric modeling such as surfaces and curves can be generated and/or used by the control electronics 54 to program and control the subsequent laser-assisted surgical procedure. The spatial disposition of the structures of interest and/or of suitable matching geometric modeling can also be used to determine a wide variety of parameters related to the procedure such as, for example, the upper and lower axial limits of the focal planes used for cutting the lens capsule and segmentation of the lens cortex and nucleus, and the thickness of the lens capsule among others.

The ranging subsystem 46 in FIG. 3 includes an OCT light source and detection device 98. The OCT light source and detection device 98 includes a light source that generates and emits light with a suitable broad spectrum. For example, in many embodiments, the OCT light source and detection device 98 generates and emits light with a broad spectrum from 810 nm to 850 nm wavelength. The generated and emitted light is coupled to the detection device 98 by a single mode fiber optic connection.

The light emitted from the OCT light source and detection device 98 is passed through a beam combiner 100, which divides the light into a sample portion 102 and a reference portion 104. A significant portion of the sample portion 102 is transmitted through the shared optics 50. A relative small portion of the sample portion is reflected from the patient interface 52 and/or the patient's eye 43 and travels back through the shared optics 50, back through the beam combiner 100 and into the OCT light source and detection device 98. The reference portion 104 is transmitted along a reference path 106 having an adjustable path length. The reference path 106 is configured to receive the reference portion 104 from the beam combiner 100, propagate the reference portion 104 over an adjustable path length, and then return the reference portion 104 back to the beam combiner 100, which then directs the returned reference portion 104 back to the OCT light source and detection device 98. The OCT light source and detection device 98 then directs the returning small portion of the sample portion 102 and the returning reference portion 104 into a detection assembly, which employs a time domain detection technique, a frequency detection technique, or a single point detection technique. For example, a frequency-domain technique can be used with an OCT wavelength of 830 nm and bandwidth of 10 nm.

Once combined with the UF laser pulse beam 66 subsequent to the beam combiner 82, the OCT sample portion beam 102 follows a shared path with the UF laser pulse beam 66 through the shared optics 50 and the patient interface 52. In this way, the OCT sample portion beam 102 is generally indicative of the location of the UF laser pulse beam 66. Similar to the UF laser beam, the OCT sample portion beam 102 passes through the Z-telescope 84 , is redirected by the X-scan device 86 and by the Y-scan device 88, passes through the objective lens assembly 94 and the patient interface 52, and on into the eye 43. Reflections and scatter off of structures within the eye provide return beams that retrace back through the patient interface 52, back through the shared optics 50, back through the beam combiner 100, and back into the OCT light source and detection device 98. The returning back reflections of the sample portion 102 are combined with the returning reference portion 104 and directed into the detector portion of the OCT light source and detection device 98, which generates OCT signals in response to the combined returning beams. The generated OCT signals that are in turn interpreted by the control electronics to determine the spatial disposition of the structures of interest in the patient's eye 43. The generated OCT signals can also be interpreted by the control electronics to measure the position and orientation of the patient interface 52, as well as to determine whether there is liquid disposed between the posterior surface of the patient interface lens 96 and the patient's eye 43.

The OCT light source and detection device 98 works on the principle of measuring differences in optical path length between the reference path 106 and the sample path. Therefore, different settings of the Z-telescope 84 to change the focus of the UF laser beam do not impact the length of the sample path for a axially stationary surface in the eye of patient interface volume because the optical path length does not change as a function of different settings of the Z-telescope 84. The ranging subsystem 46 has an inherent Z range that is related to light source and the detection scheme, and in the case of frequency domain detection the Z range is specifically related to the spectrometer, the wavelength, the bandwidth, and the length of the reference path 106. In the case of ranging subsystem 46 used in FIG. 3A, the Z range is approximately 4-5 mm in an aqueous environment. Extending this range to at least 20-25 mm involves the adjustment of the path length of the reference path 106 via a stage ZED within ranging subsystem 46. Passing the OCT sample portion beam 102 through the Z-telescope 84, while not impacting the sample path length, allows for optimization of the OCT signal strength. This is accomplished by focusing the OCT sample portion beam 102 onto the targeted structure. The focused beam both increases the return reflected or scattered signal that can be transmitted through the single mode fiber and increases the spatial resolution due to the reduced extent of the focused beam. The changing of the focus of the sample OCT beam can be accomplished independently of changing the path length of the reference path 106.

Because of the fundamental differences in how the sample portion 102 (e.g., 810 nm to 850 nm wavelengths) and the UF laser pulse beam 66 (e.g., 1020 nm to 1050 nm wavelengths) propagate through the shared optics 50 and the patient interface 52 due to influences such as immersion index, refraction, and aberration, both chromatic and monochromatic, care must be taken in analyzing the OCT signal with respect to the UF laser pulse beam 66 focal location. A calibration or registration procedure as a function of X, Y, and Z can be conducted in order to match the OCT signal information to the UF laser pulse beam focus location and also to the relative to absolute dimensional quantities.

There are many suitable possibilities for the configuration of the OCT interferometer. For example, alternative suitable configurations include time and frequency domain approaches, single and dual beam methods, swept source, etc., are described in U.S. Pat. Nos. 5,748,898; 5,748,352; 5,459,570; 6,111,645; and 6,053,613.

The system 2 can be set to locate the anterior and posterior surfaces of the lens capsule and cornea and ensure that the UF laser pulse beam 66 will be focused on the lens capsule and cornea at all points of the desired opening. Imaging modalities and techniques described herein, such as for example, Optical Coherence Tomography (OCT), and such as Purkinje imaging, Scheimpflug imaging, confocal or nonlinear optical microscopy, fluorescence imaging, ultrasound, structured light, stereo imaging, or other known ophthalmic or medical imaging modalities and/or combinations thereof may be used to determine the shape, geometry, perimeter, boundaries, and/or 3-dimensional location of the lens and lens capsule and cornea to provide greater precision to the laser focusing methods, including 2D and 3D patterning. Laser focusing may also be accomplished using one or more methods including direct observation of an aiming beam, or other known ophthalmic or medical imaging modalities and combinations thereof, such as but not limited to those defined above.

Optical imaging of the cornea, anterior chamber and lens can be performed using the same laser and/or the same scanner used to produce the patterns for cutting. Optical imaging can be used to provide information about the axial location and shape (and even thickness) of the anterior and posterior lens capsule, the boundaries of the cataract nucleus, as well as the depth of the anterior chamber and features of the cornea. This information may then be loaded into the laser 3-D scanning system or used to generate a three dimensional model/representation /mage of the cornea, anterior chamber, and lens of the eye, and used to define the cutting patterns used in the surgical procedure.

Observation of an aim beam can also be used to assist in positioning the focus point of the UF laser pulse beam 66. Additionally, an aim beam visible to the unaided eye in lieu of the infrared OCT sample portion beam 102 and the UF laser pulse beam 66 can be helpful with alignment provided the aim beam accurately represents the infrared beam parameters. The alignment guidance subsystem 48 is included in the assembly 62 shown in FIG. 3A. An aim beam 108 is generated by an aim beam light source 110, such as a laser diode in the 630-650 nm range.

Once the aim beam light source 110 generates the aim beam 108, the aim beam 108 is transmitted along an aim path 112 to the shared optics 50, where it is redirected by a beam combiner 114. After being redirected by the beam combiner 114, the aim beam 108 follows a shared path with the UF laser pulse beam 66 through the shared optics 50 and the patient interface 52. In this way, the aim beam 108 is indicative of the location of the UF laser pulse beam 66. The aim beam 108 passes through the Z-telescope 84, is redirected by the X-scan device 86 and by the Y-scan device 88, passes through the beam combiner 90, passes through the objective lens assembly 94 and the patient interface 52, and on into the patient's eye 43.

The video subsystem 92 is operable to obtain images of the patient interface and the patient's eye. The video subsystem 92 includes a camera 116, an illumination light source 118, and a beam combiner 120. The video subsystem 92 gathers images that can be used by the control electronics 54 for providing pattern centering about or within a predefined structure. The illumination light source 118 can be generally broadband and incoherent. For example, the illumination light source 118 can include multiple LEDs. The wavelength of the illumination light source 118 is preferably in the range of 700 nm to 750 nm, but can be anything that is accommodated by the beam combiner 90, which combines the light from the illumination light source 118 with the beam path for the UF laser pulse beam 66, the OCT sample portion beam 102, and the aim beam 108 (beam combiner 90 reflects the video wavelengths while transmitting the OCT and UF wavelengths). The beam combiner 90 may partially transmit the aim beam 108 wavelength so that the aim beam 108 can be visible to the camera 116. An optional polarization element can be disposed in front of the illumination light source 118 and used to optimize signal. The optional polarization element can be, for example, a linear polarizer, a quarter wave plate, a half-wave plate or any combination. An additional optional analyzer can be placed in front of the camera. The polarizer analyzer combination can be crossed linear polarizers thereby eliminating specular reflections from unwanted surfaces such as the objective lens surfaces while allowing passage of scattered light from targeted surfaces such as the intended structures of the eye. The illumination may also be in a dark-filed configuration such that the illumination sources are directed to the independent surfaces outside the capture numerical aperture of the image portion of the video system. Alternatively the illumination may also be in a bright field configuration. In both the dark and bright field configurations, the illumination light source can be used as a fixation beam for the patient. The illumination may also be used to illuminate the patient's pupil to enhance the pupil iris boundary to facilitate iris detection and eye tracking. A false color image generated by the near infrared wavelength or a bandwidth thereof may be acceptable.

The assembly 62 of system 2 may comprise a fixation light 119 that provides visible light for the patient to fixate during measurement, alignment and treatment of the eye, for example. A lens 117 can be provided to direct light to the eye 43 with vergence suitable for viewing the fixation light. Light emitted from lens 117 is reflected with beam splitter 121 along the optical path of the video camera and illumination optics.

The lens 117 may comprise a fixed lens or a variable lens, for example. The lens 117 may comprise a first configuration to provide a first optical vergence of the light entering the eye prior to placement of fluid on the eye and a second vergence subsequent placement of the interface fluid on the eye in order to correct for changes in refraction of the eye when fluid contacts the cornea. The first configuration may comprise a substantially fixed vergence, or a variable vergence adjusted to the refractive properties of the eye, for example with a variable lens. For an emmetropic patient, the light entering the eye prior to placement of the interface fluid can be collimated, for example. The second configuration of lens 117 can provide a convergent light beam to the eye to focus light onto the retina. As the cornea comprises about 40 Diopters (hereinafter “D”) of optical power, and the interface fluid can substantially decrease the optical power of the eye, the lens 117 in the second configuration may provide about 40 D of positive optical power to focus light onto the retina of the eye. This approximately 40D of positive vergence can be quite helpful with embodiments where the patient is asked to fixate on the light when the patient interface fluid has been placed on the cornea

The illumination light from the illumination light source 118 is transmitted through the beam combiner 120 to the beam combiner 90. From the beam combiner 90, the illumination light is directed towards the patient's eye 43 through the objective lens assembly 94 and through the patient interface lens 96. The illumination light reflected and scattered off of various structures of the eye 43 and patient interface travel back through the patient interface lens 96, back through the objective lens assembly 94, and back to the beam combiner 90. At the beam combiner 90, the returning light is directed back to the beam combiner 120 where the returning light is redirected toward the camera 116. The beam combiner can be a cube, plate or pellicle element. It may also be in the form of a spider mirror whereby the illumination transmits past the outer extent of the mirror while the image path reflects off the inner reflecting surface of the mirror. Alternatively, the beam combiner could be in the form of a scraper mirror where the illumination is transmitted through a hole while the image path reflects off of the mirrors reflecting surface that lies outside the hole. The camera 116 can be a suitable imaging device, for example but not limited to, any silicon based detector array of the appropriately sized format. A video lens forms an image onto the camera's detector array while optical elements provide polarization control and wavelength filtering respectively. An aperture or iris provides control of imaging NA and therefore depth of focus and depth of field and resolution. A small aperture provides the advantage of large depth of field that aids in the patient docking procedure. Alternatively, the illumination and camera paths can be switched. Furthermore, the aim beam light source 110 can be made to emit infrared light that would not be directly visible, but could be captured and displayed using the video subsystem 92.

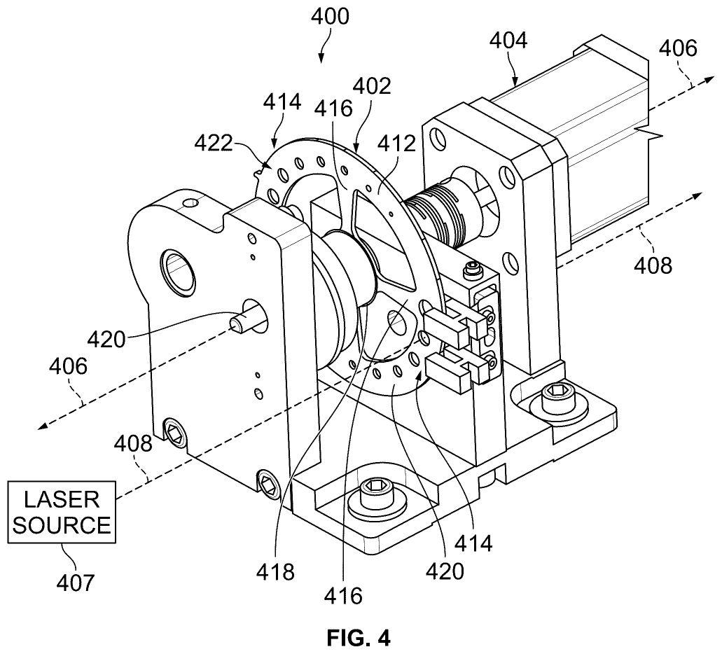

FIG. 4 illustrates a perspective view of a beam shaping or a spot control apparatus 400 for a laser surgery system, such as the system 2 of FIG. 1. The beam shaping apparatus 400 may correspond to the beam shaping apparatus 72 of FIG. 3. The beam shaping apparatus 400 includes a rotatable element or plate 402 and a drive or actuator mechanism 404. The drive mechanism 404 is configured to selectively rotate or move the rotatable element 402 about an axis of rotation 406. The drive mechanism 404 may include a stepper motor, a linear motor, a servo motor, a DC motor, or any other suitable motor or device to rotate the rotatable element 402 to various angular orientations or positions.

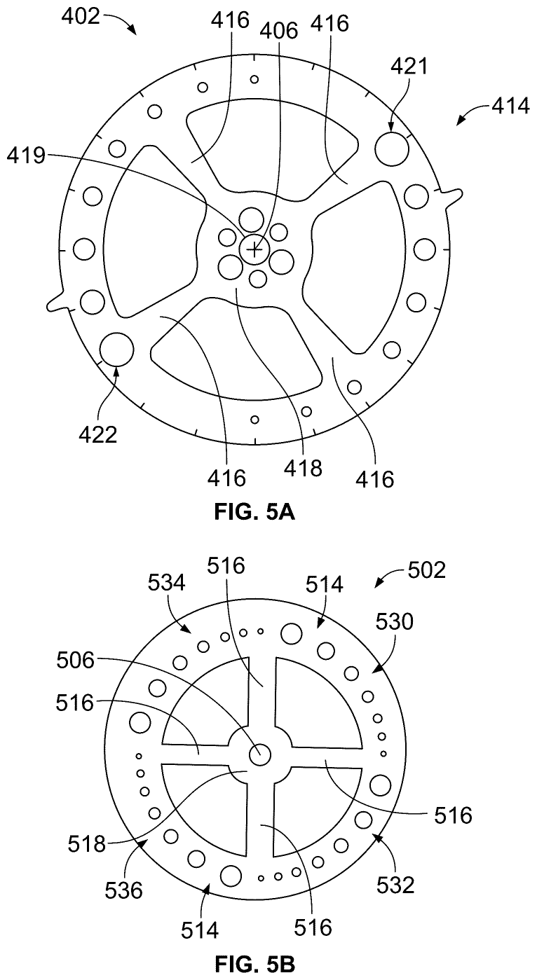

The rotatable element 402 of the beam shaping apparatus 400 is configured to control one or more of the characteristics of a laser beam generated by a laser source 407. The rotatable element 402 can be selectively and/or intermittently rotated by the drive mechanism 404 to selective angular positions about the rotational axis 406. The rotational axis 406 may be parallel to and offset from a path 408 of the laser beam generated by the laser source 407. The rotatable element 402 may comprises an aperture plate, a spot mask, a disk, a wheel, a beam reducer, or a beam profiler. The rotatable element 402 may have a substantially cylindrical or circular configuration. FIG. 5A shows a front view of the rotatable element 402 of FIG. 4. The rotatable element 402 may be formed from stainless steel, aluminum, titanium, or any other suitable material.

As shown in FIGS. 4 and 5A, the rotatable element 402 includes an outer ring portion 412, a plurality of apertures 414, interconnecting members 416, and a central or inner portion 418. The outer ring portion 412 of the rotatable element 402 is spaced radially from the central portion 418. The central portion 418 has a circular shape but it can have any suitable shape. The central portion 418 defines a central circular opening or bore 419 extending therethrough. The central circular bore is adapted to allow a shaft 420 of the drive mechanism 404 to be placed and secured thereto. The interconnecting members 416 are form and extend between the outer ring portion 412 and the central portion 418 at equal distant circumferentially spaced locations about the rotational axis 406 of the rotatable element 402.

The plurality of apertures 414 are formed on the outer ring portion 412 of the rotatable element 402 and are positioned at circumferential or angular locations about the rotation axis 406. Each of the plurality of apertures 414 is centered equally distant from the rotation axis 406 to allow each of the plurality of apertures 414 to be selectively aligned with the path 408 of the laser beam. The plurality of apertures 414 of the rotatable element 402 allow the laser beam to pass through the rotatable element 402 and are configured to change or modify characteristics of the laser beam that is passed therethrough. For example, the selected aperture may change or modify the shape, diameter, profile, and/or the intensity of the laser beam. In some embodiments, the rotatable element 402 may reduce the diameter or cross-section of the laser beam.

The rotatable element 402 of the beam shaping apparatus 400 may be intermittently and/or selectively rotated to align one of the plurality of apertures 414 with the path 408 of the laser beam such that the center of the selected aperture is substantially aligned with the center of the path 408 of the laser beam. As such, the laser beam can be passed through different ones of the plurality of apertures 414 according to a treatment procedure or plan. The rotatable element 402 can be quickly and accurately rotated to precise orientations to prevent misalignment between the selected one of the plurality of apertures 414 and the path 408 of the laser beam. Further, the rotatable element 402 may be rapidly rotated during a treatment to align an adjacent or another aperture with the path 408 of the laser beam. As a result, successive apertures may be aligned with the path 408 of the laser beam between laser beam pulses within required tolerances. For example, the rotatable element 402 can be rotated to change or switch from one aperture to an adjacent or another aperture within 20 ms and within a 20 um position tolerance. In some embodiments, one or more sensors (e.g., an encoder) may be positioned adjected to the rotatable element 402 for producing feedback signals based on the rotation or movement of the rotatable element 402. The feedback signals may be used to calibrate the drive mechanism 404 for rotating the rotatable element 402 so that a 20 um position tolerance with a 20 ms moving interval requirement can be continually met.

Each of the plurality of apertures 414 defines a respective opening or bore extending through the rotatable element 402. The plurality of apertures 414 may have various shapes, profiles, diameters, cross-sections, and/or sizes. In some embodiments, the plurality of the apertures 414 each have a substantially circular cross-sectional shape but can have a triangular shape, a cone shape, a funnel shape, a diamond shape, a hexagon shape, an elliptical shape, a non-symmetrical shape, or any other suitable shape. Each of the plurality of apertures 414 allows a portion of the laser beam to pass through the rotatable element 402 and blocks the portion of the laser beam that is greater than the dimensions of the selected aperture. As a result, each of the plurality of apertures 414 will change or modify the laser beam that passes through the rotatable element 402 to the shape or profile of the selected aperture. For example, once the laser beam passes through the rotatable element 402, the laser beam may have approximately the same cross-sectional shape or profile of the selected aperture. In some embodiments, successive pulses can be changed or reduced in diameter by the rotatable element 402 so that the laser pulses become successively smaller. In other embodiments, successive pulses can be incrementally increased from a small to large diameter.

As shown in FIGS. 4 and 5A, the plurality of apertures 414 may have a first set of apertures 421 and a second set of apertures 422. The first and second sets 421 and 422 each have a predetermined number of apertures oriented at predefined angles respective to the center or rotational axis 406 of the rotatable element 402. The apertures of the first and second sets 421 and 422 each have different diameters or cross-sections within a range equal to or smaller than the diameter or cross-section of the laser beam generated but the laser source 407. The center of each of the apertures of the first and second sets 421 and 422 may be about 35 mm from the center or rotational axis 406 of the rotatable element 402. As shown, the apertures of each set 421 and 422 are arranged in diameter or size from a large to small or small to large. In some embodiments, each of the first and second sets 421 and 422 may include eight (8) apertures and the size of the apertures for each set may be about 1.2 mm, 1.8 mm, 2.4 mm, 3.0 mm. 3.6 mm, 4.2 mm, 4.8 mm, and 7.0 mm, respectfully. Each of the apertures of the first and second sets 421 and 422 may be separated by a predetermined angle. For example, the angle between the center of the apertures of each set 421 and 422 may be about 18 degrees.

Referring now to FIG. 5B, another exemplary embodiment of a rotatable element 502 of a beam shaping apparatus is shown. The beam shaping apparatus may correspond to the beam shaping apparatus 72 of FIG. 3. The rotatable element 502 has a circular shape or configuration with uniform thickness. The rotatable element 502 includes an outer ring portion 512, a plurality of apertures 514, interconnecting members 516, and a central or inner portion 518. As described above, the plurality of apertures 514 of the rotatable element are configured to change or modify a characteristic of the laser beam that is passed through the rotatable element 502. For example, the plurality of apertures 514 may change or modify the shape, diameter, cross-section, profile, and/or intensity of the laser beam. In some embodiments, the rotatable element 502 may reduce the diameter or cross-section of the laser beam.

As shown in FIG. 5B, the plurality of apertures 515 may include four sets of apertures 530, 532, 534, and 536. Each set of apertures 530, 532, 534, and 536 has a predetermined number of apertures oriented at predetermined angles respective to the center or rotational axis 506 of the rotatable element 502. The apertures of each set 530, 532, 534, and 536 have different diameters or cross-sections and are arranged in size or diameter from a large to small or small to large. The plurality of apertures of each set 530, 532, 534, and 536 may have a substantially circular shape but can have a triangular shape, a cone shape, a funnel shape, a diamond shape, a hexagon shape, an elliptical shape, a non-symmetrical shape, or any other suitable shape.

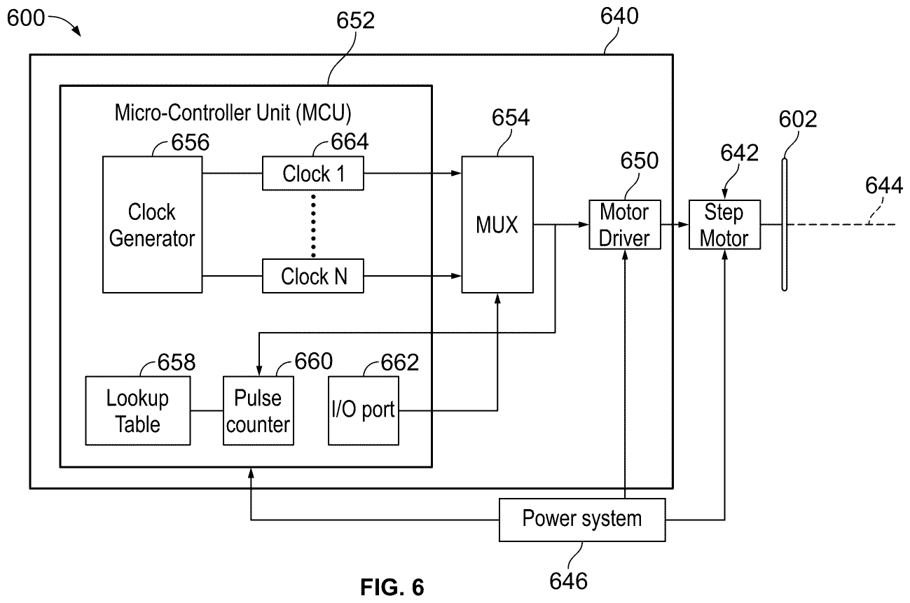

FIG. 6 illustrates a simplified block diagram of a control system or unit 600 for controlling a beam shaping apparatus of a laser surgical system, such as the system 2 of FIG. 1. The beam shaping apparatus may correspond to the beam shaping apparatus 72 of FIG. 3. The control system 600 is configured to control the rotation or movement of a rotatable element 602 of the beam shaping apparatus. The control system 600 can cause one of a plurality of apertures of the rotatable element 602 to be aligned with a path of a laser beam to allow the laser beam to pass through the rotatable element 602 and change or modify a characteristic of the laser beam.

As shown in FIG. 6, the control system 600 includes an electronic control unit or a controller 640 and a driving mechanism or actuator 642. The electronic control unit 640 is configured to generate signals or commands to drive or control the operation of the drive mechanism 642. The drive mechanism 642 includes a shaft for rotating the rotatable element 602 about an axis of rotation 644. The drive mechanism 642 may include a stepper motor, a linear motor, a servo motor, a DC motor, or any other suitable motor or device to rotate the rotatable element 602. A power system 646 supplies power to the drive mechanism 642 and to the electronic control unit 640.

The electronic control unit 640 of the control system 600 may use an open-loop driving approach with variable frequency pulse trains (e.g., a drive profile having multiple accelerate rates and decelerate rates) to start and stop the rotation of a shaft of the drive mechanism 642 (e.g., a stepper motor) to prevent drive mechanism 642 from lose-step or over-step. The frequency pulse trains may include a plurality of pulses with two or more pulses having a different width, frequency, duty cycle, and duration. The electronic control unit 640 includes a motor driver 650, a controller or processor 652, and a multiplexer 654. The controller 652 of the electronic control unit 640 may comprise a microcontroller having a clock generator 656, a look-up table 658, a pulse counter 660, and a I/O port 662. In some embodiments, the controller 652 may comprise a Renesas Synergy S7G2 micro-control unit (MCU). In other embodiments, the controller 652 may comprise a field-programmable gate array (FPGA) and, in some embodiments, the functions of the multiplexer 654 and the motor driver 650 may also be implemented in the FPGA.

The clock generator 656 of the controller 652 may generate different frequency pulse trains 664 (e.g. clock signals). In some embodiments, the clock generator 656 may comprise a 14 channel 32-bit general PWM timer (GPT). The multiplexer 654 of the electronic control unit 640 may receive the different frequency pulse trains 664 (e.g., clock signals) as inputs from the clock generator 656 of the controller 652. The multiplexer 654 may select one of the different frequency pulse trains 664 for driving the drive mechanism 642 based on at least the output of the I/O port 662 of the controller 652. The multiplexer 654 may output the selected frequency pulse train 664 as an input to the motor driver 650. The motor driver 650 may use the selected frequency pulse train 664 as the driving profile or signal for controlling the operation of the drive mechanism 642 (e.g., stepper motor) for rotating or moving the rotatable element 602 between various angular orientations. In some embodiments, S-curves drive profile may be used to drive the speed of the drive mechanism 642.

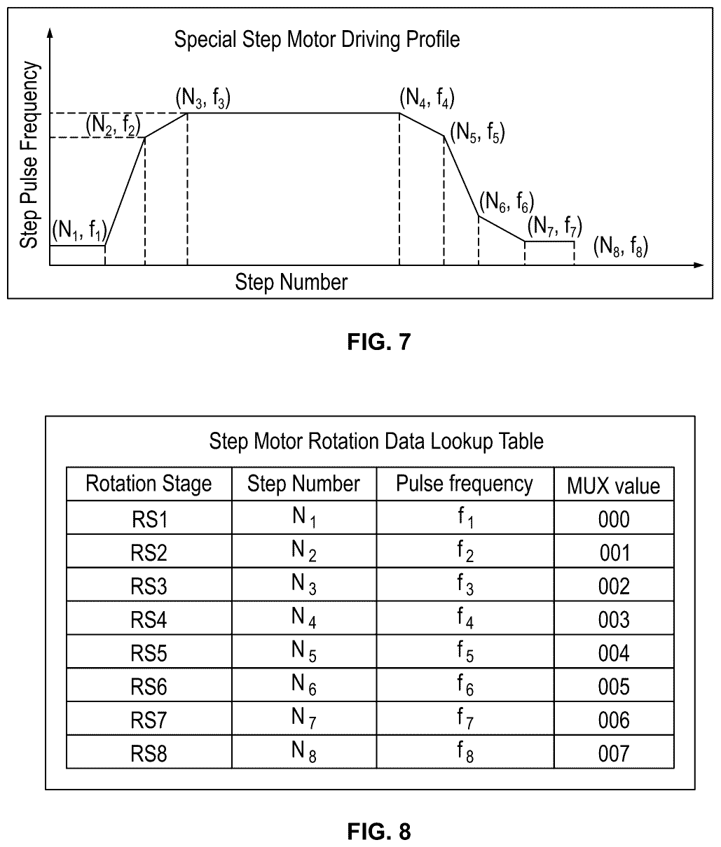

FIG. 7 shown an exemplary drive profile for controlling the drive mechanism 642. As shown in FIG. 7, the rotation stages, RS2, and RS3 of the driving profile have different acceleration steep rates, while the rotation stages RS5, RS6, and RS7, of the driving profile has different deceleration steep rates. The rotation stages RS1, RS4, and RS8 have zero acceleration for operating the drive mechanism 642 (e.g., stepper motor) at constant speeds. As such, the rotation stages, RS2 and RS3 have increasing pulse train frequency and the rotation stages RS5, RS6, and RS7, have decreasing pulse train frequency. To achieve a 20 ms moving time interval from one aperture position to an adjacent or another aperture position from start to stop, the drive profile of FIG. 7 with a 32KHz pulse train frequency at rotation stage RS4 may be used.

Referring again to FIG. 6, the output of the multiplexer 654 is also input to the pulse counter 660 of the controller 652. In some embodiments, the pulse counter 660 may comprise a 16-bit asynchronous general-purpose timer (AGT) of the controller 652. Based on the value of the pulse counter 660, rotation data corresponding to the pulse counter value may be retrieved from a look-up table 658 (LUT) of the controller 652. FIG. 8 show a representation of rotation data stored in the look-up table for controlling the operation of the drive mechanism 642.

To ensure that there is no lose-step or over-step occurring while rotating the drive mechanism 642 (e.g., a stepper motor) from start to stop positions between two apertures (e.g., adjacent apertures) within 20 ms time interval requirement, a calibration approach per motor load pull-in and pull-out torques may be used For example, one or more sensors (e.g., encoder) may be configured to produce feedback signals from the rotatable element 602 in order to calibrate the driving mechanism 604 (e.g., stepper motor). After calibration, the calibrated step number, pulse train frequency as well as the multiplexor values may be stored in the LUT 658 of the controller 652 as the data for driving mechanism 654 (e.g., a stepper motor driving process). For example, the LUT 658 may be updated or modify based on the calibration data in order maintain a 20 um position tolerance with a 20 ms moving interval specification requirement.

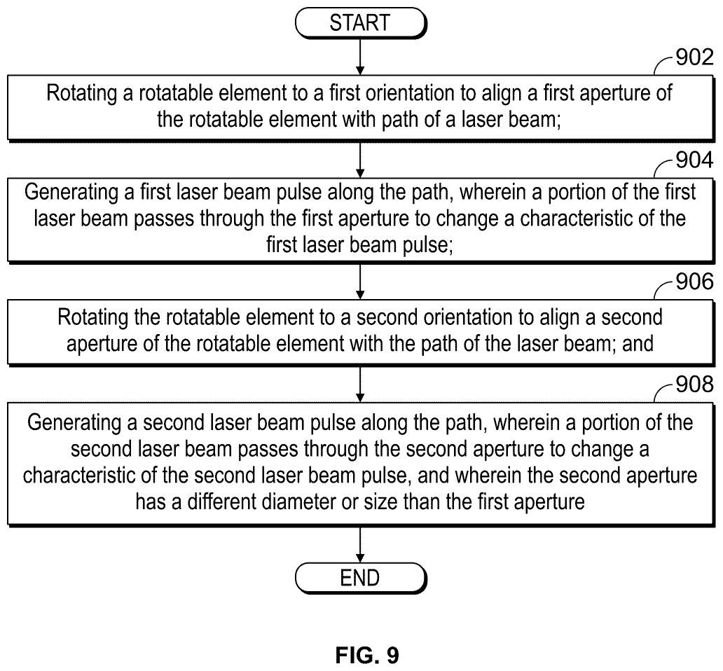

FIG. 9 is a flow chart of a method 900 of performing an ophthalmic surgical procedure, according to an example implementation. The method 900 represents an example method that may include one or more operations as depicted by one or more blocks 902-908, each of which may be carried out by any of the systems shown in FIGS. 1-4, among other possible systems. In an example implementation, a computing system (e.g., system 2) performs the illustrated operations, although in other implementations, one or more other systems can perform some or all of the operations.

Those skilled in the art will understand that the flow charts described herein illustrates functionality and operations of certain implementations of the present disclosure. In this regard, each block of the flowcharts may represent a module, a segment, or a portion of program code, which includes one or more instructions executable by one or more processors for implementing specific logical functions or steps in the processes. The program code may be stored on any type of computer readable medium, for example, such as a storage device including flash memory, EEPROM, a disk or hard drive.

In addition, each block may represent circuitry that is wired to perform the specific logical functions in the processes. Alternative implementations are included within the scope of the example implementations of the present application in which functions may be executed out of order from that shown or discussed, including substantially concurrent or in reverse order, depending on the functionality involved, as would be understood by those reasonably skilled in the art. Within examples, any system may cause another system to perform one or more of the operations (or portions of the operations) described below.

In line with the discussion above, a laser surgical system may operate as shown by method 900. The laser surgical system may include a laser source for generating a laser beam for treating a target surface, such as a human eye. The laser beam can be directed to the target surface for forming an incision, performing tissue ablation, and cauterization and/or coagulation. As described above, a beam shaping apparatus can be used to control the characteristics of the laser beam delivered to the target surface. For example, the beam shaping apparatus may control the shape, size, diameter, cross-section, profile, and/or the intensity of the laser beam. The beam shaping apparatus can include a rotatable element having a plurality of apertures of various sizes and/or shapes. The rotatable element may be rotatable or movable to align different ones of the apertures with the path of the laser beam to change or modify a characteristic of the laser beam.

At block 902, the method involves rotating a rotatable element to a first orientation to align a first aperture of the rotatable element with a path of a laser beam. At block 904, the method involves generating a first laser beam pulse along the path. A portion of the first laser beam may pass through the first aperture to change a characteristic of the first laser beam pulse.

At block 906, the method involves rotating the rotatable element to a second orientation to align a second aperture of the rotatable element with the path of the laser beam. At block 908, the method involves generating a second laser beam pulse along the path. A portion of the second laser beam may pass through the second aperture to change a characteristic of the second laser beam pulse. Further, the second aperture may have a different diameter or size than the first aperture.

The present disclosure has been described in terms of specific embodiments incorporating details to facilitate the understanding of principles of construction and operation of the embodiments. Such reference herein to specific embodiments and details thereof is not intended to limit the scope of the claims appended hereto. It will be apparent to those skilled in the art that modifications may be made in the embodiment chosen for illustration without departing from the spirit and scope of the disclosure. Although the system and methods of the present disclosure are described primarily in a context of a laser eye surgery system, it should be understood techniques of the present disclosure may be adapted for use in alternative eye treatment procedures and systems such as such as orthopedic surgery, robotic surgery, and microkeratomes. and the like.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Although features and elements are described above in particular combinations, one of ordinary skill in the art will appreciate that each feature or element can be used alone or in any combination with the other features and elements. In addition, the methods described herein may be implemented in a computer program, software, or firmware incorporated in a computer-readable medium for execution by a computer or processor. A computer readable medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Examples of computer-readable media include electrical signals (transmitted over wired or wireless connections) and computer-readable storage media. Examples of computer-readable storage media include, but are not limited to, a register, cache memory, semiconductor memory devices, magnetic media such as internal hard disks and removable disks, magneto-optical media, optical media such as compact disks (CD) and digital versatile disks (DVDs), a random access memory (RAM), a read-only memory (ROM), an erasable programmable read-only memory (EPROM or Flash memory), a static random access memory (SRAM), and a memory stick. A processor in association with software may be used to implement a radio frequency transceiver for use in a terminal, base station, or any host computer.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one more other features, integers, steps, operations, element components, and/or groups thereof.

The descriptions of the various embodiments herein have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. An apparatus for ophthalmic laser surgery comprising:
a laser source configured to generate a laser beam;
a beam shaping apparatus having a rotatable element positioned in a path of the laser beam, the rotatable element having a plurality of apertures and rotatable about an axis of rotation;
a control unit configured to generate one or more frequency pulse trains; and
a motor configured to rotate the rotatable element to align one of the plurality of apertures with the path of the laser beam for changing or modifying a characteristic of the laser beam, wherein the motor is driven based on the one or more frequency pulse trains.

2. The apparatus of claim 1, wherein the rotatable element comprises an aperture plate, a spot mask, a disk, a wheel, a beam reducer, or a beam profiler.

3. The apparatus of claim 1, wherein the one of the plurality of apertures is configured to enable at least a portion of the laser beam from the laser source to pass through the rotatable element.

4. The apparatus of claim 1, wherein the characteristic of the laser beam comprises a shape, a size, a diameter, a dimension, or an intensity.

5. The apparatus of claim 1, wherein the plurality of apertures are spaced angularly around a periphery of the rotatable element.

6. The apparatus of claim 1, wherein each of the plurality of apertures of the rotatable element has a substantially circular or cone shape.

7. The apparatus of claim 1, wherein each of the plurality of the apertures of the rotatable element has a shape, wherein the shape for each of the plurality of apertures comprises a triangle, a diamond, a hexagon, a circle, an ellipse, a cone, a funnel, or a non-symmetrical shape.

8. The apparatus of claim 1, wherein the plurality of apertures of the rotatable element includes a first set of apertures and a second set of apertures, wherein apertures of the first set each have a different diameter and are disposed at equal angles around a periphery of the rotatable element.

9. The apparatus of claim 8, wherein the apertures of the first set are arranged from small to large in diameter or size.

10. The apparatus of claim 1, wherein the rotatable element is formed from stainless steel, aluminum, or titanium.

11. The apparatus of claim 1, wherein the axis of rotation is parallel to and offset from the path of the laser beam, and wherein the rotatable element is rotated in a plane perpendicular to the path of the laser beam.

12. The apparatus of claim 1, wherein the control unit is further configured to:
select a first frequency pulse train from the one or more frequency pulse trains for driving the motor; and
provide the first frequency pulse train to the motor to cause the rotatable element to rotate by a predetermined angle to a different angular orientation, wherein the first frequency pulse train includes different acceleration pulses to increase motor speed, and wherein the different acceleration pulses include at least two pulses differing in at least one of width, frequency, duty cycle, and duration.

13. The apparatus of claim 12, wherein the first frequency pulse train includes different deceleration pulses for decreasing motor speed, and wherein the different deceleration pulses include at least two pulses differing in at least one of width, frequency, duty cycle, and duration.

14. The apparatus of claim 1, wherein the motor comprises a stepper motor.

15. The apparatus of claim 1, wherein the control unit is further configured to select one of the one or more frequency pulse trains based on data stored in a look-up table, and wherein data is selected from the look-up table based on a value of a pulse counter of a controller.

16. A method of performing an ophthalmic surgical procedure comprising:
rotating a rotatable element to a first orientation to align a first aperture of the rotatable element with a path of a laser beam;
generating a first laser beam along the path, wherein a portion of the first laser beam passes through the first aperture to change a characteristic of the first laser beam;
rotating the rotatable element to a second orientation to align a second aperture of the rotatable element with the path of the laser beam, wherein the rotatable element is rotated based on one or more frequency pulse signals; and
generating a second laser beam along the path, wherein a portion of the second laser beam passes through the second aperture to change a characteristic of the second laser beam, and wherein the second aperture has a different diameter or size than the first aperture.

17. The method of claim 16, wherein the characteristic of the first laser beam comprises a shape, a size, a diameter, a dimension, or intensity, and wherein the characteristic of the second laser beam comprises a shape, a size, a diameter, a dimension, or an intensity.

18. The method of claim 16, wherein the rotatable element is rotated using a stepper motor.

19. The method of claim 16, wherein the first and second apertures are positioned along a periphery of the rotatable element.

20. The method of claim 16, wherein the rotatable element is rotated from the first orientation to the second orientation in response to a first frequency pulse signal of the one or more frequency pulse signals, wherein the first frequency pulse signals includes a first plurality of pulses to cause the rotatable element to accelerate during a first time period, and wherein each of the pulses of the first plurality of pulses differ in at least one of width, frequency, duty cycle, and duration, wherein the first frequency pulse signals further includes a second plurality of pulses to cause the rotatable element to decelerate during a second time period, and wherein each of the pulses of the second plurality of pulses differ in at least one of width, frequency, duty cycle, and duration.
